(19) United States
(12) Patent Application Publication
(10) Pub. No.: US 2024/0248463 A1
(43) Pub. Date: Jul. 25, 2024

(54) AI ENHANCED, SELF CORRECTING AND CLOSED LOOP SMT MANUFACTURING SYSTEM

(71) Applicant: JABIL INC., St. Petersburg, FL (US)

(72) Inventors: Anwar A. Mohammed, St. Petersburg, FL (US); Harpuneet Singh, St. Petersburg, FL (US); Nicholas Randall Tokotch, St. Petersburg, FL (US); Gary Rojo, St. Petersburg, FL (US); Bonnie Lowell, St. Petersburg, FL (US)

(21) Appl. No.: 18/560,535

(22) PCT Filed: May 11, 2022
(86) PCT No.: PCT/US2022/072243

§ 371 Date: Nov. 13, 2023

Related U.S. Application Data
(60) Provisional application No. 63/187,012, filed on May 11, 2021.

(51) Int. Cl. G05B 19/418; H05K 3/12

(52) U.S. Cl. CPC G05B 19/41875; H05K 3/1233; G05B 2219/32194; G05B 2219/37217

(57) ABSTRACT

An AI enhanced self-correcting and closed loop SMT manufacturing system for fabricating PCBAs. The system includes a screen printer for depositing solder paste on solder pads on a RGB, an SRI sub-system for inspecting the solder paste deposited on the PCB to identify defects, a pick-and-place machine for placing circuit components on the solder paste, an AOI sub-system for inspecting the PCB after the circuit components are placed on the PCB, and a reflow soldering oven for bonding component leads both electrically and mechanically to the pads on the PCB. An AI/ML analysis engine is responsive to process data and variables from each of the screen printer, the SPI sub-system, the pick-and-place machine, the AOI sub-system and the reflow soldering oven and provides downstream feedback signals to each of the screen printer, the SPI sub-system, the pick-and-place machine, the AOI sub-system and the reflow soldering oven for self-correction purposes.

### BACKGROUND

### Field

This disclosure relates generally to an artificial intelligence (AI) enhanced, self-correcting and closed loop surface mount technology (SMT) manufacturing system for fabricating printed circuit board assemblies (PCBAs) and, more particularly, to an AI enhanced, self-correcting and closed loop SMT manufacturing system for fabricating PCBAs, where the system includes a process analysis engine that employs an AI/machine learning (ML) model to provide process feedback control for self-correcting purposes.

### Discussion

SMT refers to a technique for fabricating electronic circuits where the components of the circuit are electrically mounted or placed directly on the surface of a PCB to produce a PCBA. The PCB is generally a flat dielectric board having a surface on which is formed tin-lead, silver or gold plated copper pads that do not have holes, known as solder pads, in a predetermined configuration. A solder paste, which is a sticky mixture of solder flux and solder particles or flakes, is deposited on the solder pads by using a stainless steel or nickel stencil and a screen printing process, but can also be applied by a jet-printing mechanism, such as an inkjet printer, where it is critical that the solder paste be accurately oriented to the solder pad to prevent short circuits and the like.

The PCB is then placed on a conveyor belt to be sent to a pick-and-place machine. The components to be mounted on the PCB are usually delivered to the pick-and-place machine on either a paper/plastic tape wound on a reel or a plastic tube, where large integrated circuits can be delivered to the pick-and-place machine on static-free trays. The pick-and-place machine removes the components from the tape, tube or tray and properly places them on the solder pads on the PCB in a predetermined manner, where the components are held in place by the tackiness of the solder paste. The PCB is then sent to a reflow soldering oven that includes a pre-heat zone, where the temperature of the PCB is gradually and uniformly raised. The PCB then enters a high temperature zone where the temperature is high enough to melt the solder particles in the solder paste, such as 260° C., which bonds the component leads to the solder pads on the PCB. The surface tension of the molten solder helps keep the components in place, and if the solder pad geometries are correctly designed, the surface tension automatically aligns the components on their pads.

It is known that most of the solder joint defects that occur in a PCBA are caused by improper solder paste printing. Therefore, SMT processes often employ a solder paste inspection (SPI) system to inspect the solder paste deposits on the PCB in order to identify the volume of the solder paste and the x, y and z orientation of the solder paste relative to the solder pads, i.e., the volumetric center of the solder paste is where it should be located, to reduce PCB defects. As the pitch of the components becomes more fine, i.e., the number of components on the same area of the PCB increases and the leads of the components become closer together, the exact position of the solder paste becomes more critical to prevent short circuits. Such SPI systems typically include an arrangement of cameras and other sensing devices to obtain a visual image of the solder paste on the PCB to provide the inspection.

However, known SPI systems used in SMT processes are limited in their capabilities. For example, known SPI systems are generally not able to identify the pitch of the components, i.e., the spacing between the components, where a higher pitch of the components may require a slower inspection speed. Another drawback with the known SPI systems is that they do not provide key printing variables such as temperature and humidity, which can change during the SMT process and can be used to determine the viscosity of the solder paste, where the viscosity identifies the rheology of the solder paste, which determines how well the solder paste will go through the stencil and stay on the solder pad. Also, the known SPI systems are typically not able to identify the type of solder flux in the solder paste to verify whether the correct solder flux is being used, or identify the type of solder or the size of the solder flakes being used. Currently, solder flux is color coded to identify it, but the known SPI systems cannot identify that color. All of the viscosity of the solder paste, the type of solder flux, the type of solder and the size of the solder flakes can be used to determine if the proper stencil or screen is being used.

Automated optical inspection (AOI) is an automated non-contact visual inspection process of circuit devices, such as PCBAs fabricated by SMT processes, where a camera autonomously scans the PCBA to monitor for catastrophic failure, such as missing parts, and quality defects, such as solder flow issues. However, known AOI processes for SMT are also limited in their capabilities. For example, known AOI processes do not determine the presence or measure the volume of inter-metallic compounds (IMCs), i.e., undesirable materials that are generated by the type of solder and the solder flow process, which could affect the electrical connection of the component leads to the solder pads and cause a reliability issues. Further, known AOI systems do not determine whether voids exist between the flowed solder and the solder pads, which also could affect thermal and electrical bond integrity. Specifically, if the voids between the flowed solder and the solder pads are numerous enough or large enough, power dissipation, i.e., heat removal, may be effected, especially for high pitch components. Also, known AOI systems do not determine whether the flowed solder is planar relative to the solder pads, i.e., the slope of the soldered bond line thickness (BLT), which limits its ability to be wire-bonded.

Variations of SMT manufacturing processes often result in undesirable post-reflow component conditions during PCB reflow that fail SMT workmanship quality standards, commonly referred to as SMT manufacturing defects. These SMT defects have a significant impact on product quality and manufacturing costs due to the waste associated with scrap, rework, downtime and other non-value add activities.

### SUMMARY

This disclosure discloses and describes an AI enhanced, self-correcting and closed loop SMT manufacturing system for fabricating PCBAs. The system includes a screen printer for depositing solder paste on a conductive solder pads on a PCB, and an SPI sub-system for inspecting the solder paste deposited on the PCB to identify defects. The system further includes a pick-and-place machine for placing circuit components on the solder paste, an AOI sub-system for inspecting the PCB after the circuit components are placed on the PCB, and a reflow soldering oven for bonding component leads both electrically and mechanically to the pads on the PCB. An AI/ML analysis engine is responsive to process data and variables from each of the screen printer, the SPI sub-system, the pick-and-place machine, the AOI sub-system and the reflow soldering oven and provides downstream feedback signals to each of the screen printer, the SPI sub-system, the pick-and-place machine, the AOI sub-system and the reflow soldering oven for self-correction purposes.

Additional features of the disclosure will become apparent from the following description and appended claims, taken in conjunction with the accompanying drawings.

### BRIEF DESCRIPTION OF THE DRAWING

FIG. 1 is a simplified block diagram of an AI enhanced, self-correcting and closed loop SMT manufacturing system for fabricating PCBAs.

### DETAILED DESCRIPTION OF THE EMBODIMENTS

The following discussion of the embodiments of the disclosure directed to an AI enhanced, self-correcting and closed loop SMT manufacturing system for fabricating PCBAs is merely exemplary in nature, and is in no way intended to limit the disclosure or its applications or uses.

This disclosure proposes an AI enhanced, self-correcting and closed loop SMT manufacturing system that enables real time trend analysis and predictive capabilities, with automatic feedback control loops and machine-to-machine dialoging, for anticipating and self-correcting potential yield losses in SMT manufacturing processes. The analysis employs a self-learning Markov decision process (MDP) model to manage sequential decision process outcomes in which states and transitions are quantified in calculated rewards during the transition between two Markov states and provide multi-agent reinforcement learning. The SMT manufacturing system provides a number of desirable features. One feature of the system includes the predictive and preventative analytics for maintaining an in-control SMT process by interlinking SMT process and measurement data across all SMT equipment. Another feature of the system includes full SMT process characterization through multi-process data correlation and verification to identify critical parameters and measurements that are significant contributors to end of line SMT quality. Another feature of the system includes real time SMT process monitoring, process control and process correction using smart algorithms leveraging AI capabilities for predictive modeling machine intelligence for self-correction. Yet another feature of the system includes providing a prediction and self-correction of possible SMT defects before reflow soldering results in waste due to rework and scrap. Also, the system eliminates the requirement for human intervention, decision and action with automatic machine-to-machine communication.

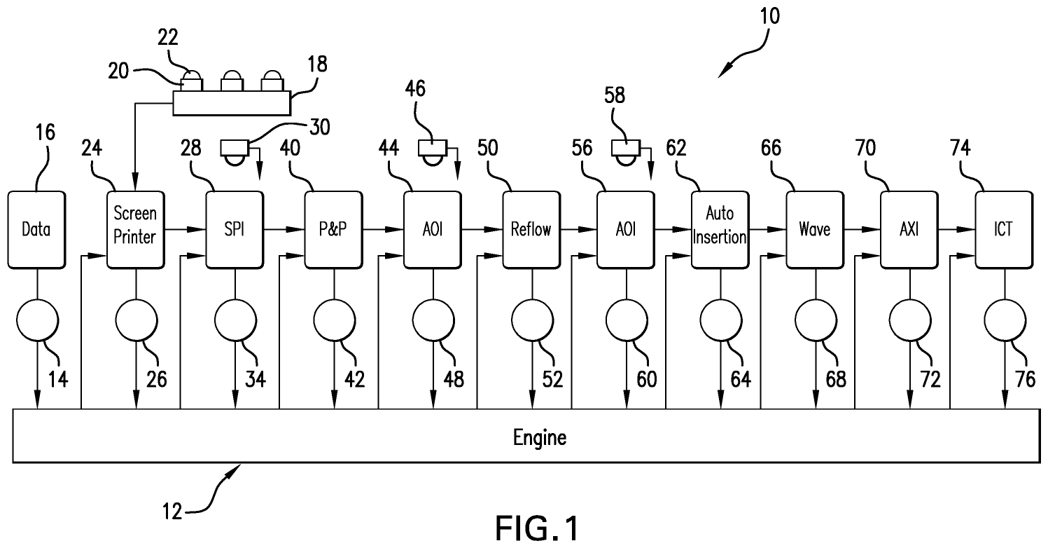

FIG. 1 is a simplified block diagram of an AI enhanced, self correcting and closed loop SMT manufacturing system 10 for fabricating PCBAs that includes an AI/ML analysis engine 12. The system 10 is intended to represent any suitable circuit fabrication system consistent with the discussion herein. The engine 12 employs an MDP model that operates as a lossless abstraction algorithm that compares the behavior abstraction of an SMT process model with finite event log behavior. The engine 12 provides learned phenomenon for comparing finite event log behavior with infinite SMT process model behavior to determine (predict) potential yield loss outcome. The engine 12 includes a component rejection prediction model that uses a multi-regression analysis ensemble model solution. The AI/ML model operating in the engine 12 accepts basic material and process data from a screen printer, provides critical measurement data from numerous inspection systems, correlates and characterizes optimal process tolerance window, provides prognostic and predictive conditions for PCB quality issues, and provides closed loop optimization commands back to process steps to maintain an in-control processes.

Raw material data and environmental conditions 14, such as PCB surface finish, PCB thickness, etc., are provided at box 16 to the engine 12 for a panel 18 including an array of PCBs 20 having conductive solder pads 22 on a top surface thereof being processed.

The panel 18 is provided to a screen printer 24 and is subjected to a printing process for depositing a solder paste, i.e., a mixture of solder flux and solder particles or flakes, on the solder pads 22 using, for example, a stainless steel or nickel stencil or screen by known processes. The screen printer 24 provides process data and variables 26, such as solder paste type, cleaning cycle stroke, screen printer parameters, etc. to the engine 12 and the engine 12 provides feedback, such as pressure adjustments, squeegee changes, stencil cleaning, etc. determined from upstream processes and inspections for screen printing self-correction to the screen printer 24.

The panel 18 is then sent to an SPI sub-system 28 to inspect the solder paste deposited on the PCBs 20 and identify any defects or other issues that would reduce PCB reliability. The SPI sub-system 28 includes an array of cameras that obtain visual images of the solder joints on the PCBs 20 and/or other sensing devices, such as a temperature sensor and a humidity sensor, represented generally by device 30. Images from the cameras and devices 30 and measurement data 34, such as solder paste offset measurements, are provided to the engine 12 that processes the signals to provide inspection information. The inspection information can include identifying the pitch or resolution of the components that may require slower inspection speeds, and using temperature and humidity measurements to determine the viscosity of the solder paste to obtain its rheology. The cameras have a resolution and image quality that allows the cameras to provide images that allow the engine 12 to identify the solder flux in the solder paste by its color, identify the type of solder in the solder paste by its color, and identify the size of the solder flakes in the solder paste. All of this information can be used to determine if the proper solder is being used and the proper screen is being used for the PCBs 20 currently being fabricated. The engine 12 provides feedback from upstream processes and inspections to the SPI sub-system 28. The feedback may require that the inspection process be slowed down, and thus the SPI sub-system 28 can alter its inspection speed on the fly as needed. The SPI sub-system 28 will enable SMT manufacturers to produce PCBAs with enhanced reliability and yields and also minimize any errors caused by using the wrong solder or flux, prevent any printing errors caused by viscosity, temperature or humidity and better detect any printing errors on fine pitch components.

If the panel 18 passes the SPI process and is not scrapped, the panel 18 is delivered to a pick-and-place machine 40 for placing circuit components on the solder paste. Particularly, the components are delivered on a tape and are picked off of the tape by the machine 40 and placed on the proper solder paste in a predetermined manner, where the components are held in place by the tackiness of the solder paste. The machine 40 provides process data and variables 42, such as ground, package and machine information, to the engine 12 and the engine 12 provides feedback from upstream processes and inspections, such as change nozzle or feeder, adjust part definition, change placement position, optimize placement offset for better placement, perform maintenance, etc., to the machine 40 for self-correction purposes. Thus, if the engine 12 determines that the location of all of the solder pastes are off-set by a certain distance, the machine 40 can receive this information and adjust the location that it drops the components accordingly.

The panel 18 now with the components on the PCBs 20 is then sent to an AOI sub-system 44 including one or more sophisticated cameras 46 or other vision devices. Images 48 from the cameras 46 and other information, such as full component condition, component off-set measurements, etc., are sent to the engine 12. The resolution and quality of the cameras 46 is such that the images 48 can identify or detect the presence and volume of inter-metallic compounds in the flowed solder between the component and the solder pads, which can provide an indication of the quality of the solder bond. The engine 12 can detect the presence and size of voids between the flowed solder and the solder pads from the images 48 to determine the thermal capability, i.e., heat removal, of the PCBs 20. Also, the engine 12 can detect the slope of the soldered bond line thickness (BLT) from the images 48, which allows for better wire bonding. The engine 12 provides feedback, such as adjust pre-flow program parameter settings to detect specific condition alerted at post-AOI, from upstream processes and inspections to the AOI sub-system 44 for self-correction purposes.

The panel 18 is then sent to a reflow soldering oven 50, where the temperature in the oven 50 is high enough to melt the solder particles in the solder paste, which bonds the component leads both electrically and mechanically to the pads 22 on the PCBs 20. The surface tension of the molten solder helps keep the components in place, and if the solder pad geometries are correctly designed, surface tension automatically aligns the components on their solder pads. The oven 50 provides process data and variables 52 to the engine 12 and the engine 12 provides feedback from upstream processes and inspections to the oven 50 for self-correction purposes.

The panel 18 is then sent to another AOI sub-system 56 including one or more sophisticated cameras 58 or other vision devices that operates in the same manner as the sub-system 44, and provides data 60, such as post reflow SMT quality condition, to and receives feedback, such as trigger post-reflow AOI inspection based on predicted post-reflow defects, from the engine 12 for self-correction purposes.

The panel 18 is then sent to an auto-insertion machine 62 that inserts additional components on the PCBs 20 that are not able to be placed by the pick-and-place machine 40, where the machine 62 provides data 64 to and receives feedback from the engine 12 for self-correction purposes.

The panel 18 is then sent to a wave solder machine 66 that provides a bulk soldering process on the PCBs 20 that is mainly used in soldering of through hole components, where the machine 66 provides data 68 to and receives feedback from the engine 12 for self-correction purposes.

The panel 18 is then sent to an in-line X-ray inspection machine 70 that performs an X-ray inspection process on the PCBs 20 to provide a high speed, solder coverage test for hidden joints, where the machine 70 provides data 72 to and receives feedback from the engine 12 for self-correction purposes. Ball grid arrays (BGA), quad flat no-lead (QFN) packages and plated through hole (PTH) barrel fill items are generally inspected during the X-ray inspection process based on the Institute of Printed Circuits (IPC) acceptance criteria.

The panel 18 is then sent to an in-circuit testing machine 74 provides electrical testing on the PCBs 20, where the machine 74 provides data 76 to and receives feedback from the engine 12 for self-correction purposes.

The foregoing discussion discloses and describes merely exemplary embodiments of the present disclosure. One skilled in the art will readily recognize from such discussion and from the accompanying drawings and claims that various changes, modifications and variations can be made therein without departing from the spirit and scope of the disclosure as defined in the following claims.

What is claimed is:

1. A surface mount technology (SMT) manufacturing system for fabricating printed circuit board assemblies (PCBAs), said system comprising:
a screen printer for depositing solder paste on conductive solder pads on a printed circuit board (PCB);
a solder paste inspection (SPI) sub-system for inspecting the solder paste deposited on the PCB to identify defects;
a pick-and-place machine for placing circuit components on the solder paste;
a first automated optical inspection (AOI) sub-system for inspecting the PCB after the circuit components are placed on the PCB;
a reflow soldering oven for bonding component leads both electrically and mechanically to the pads on the PCB; and
an artificial intelligence (AI)/machine learning (ML) analysis engine responsive to process data and variables from each of the screen printer, the SPI sub-system, the pick-and-place machine, the first AOI sub-system and the reflow soldering oven and providing feedback signals to each of the screen printer, the SPI sub-system, the pick-and-place machine, the first AOI sub-system and the reflow soldering oven for self-correction purposes.

2. The system according to claim 1 wherein the analysis engine employs a self-learning Markov decision process (MDP) model that manages sequential decision process outcomes in which states and transitions are quantified in calculated rewards during the transition between two Markov states and provides multi-agent reinforcement learning.

3. The system according to claim 1 further comprising a second AOI sub-system for inspecting the PCB after the PCB has been to the reflow soldering oven, said analysis engine being responsive to data and variables from the second AOI sub-system and providing feedback signals to the second AOI sub-system for self-correction purposes.

4. The system according to claim 1 further comprising an auto-insertion machine for inserting additional components on the PCB that are not able to be placed by the pick-and-place machine, said analysis engine being responsive to data and variables from the auto-insertion machine and providing feedback signals to the auto-insertion machine for self-correction purposes.

5. The system according to claim 1 further comprising a wave solder machine for bulk soldering the PCB, said analysis engine being responsive to data and variables from the wave soldering machine and providing feedback signals to the wave soldering machine for self-correction purposes.

6. The system according to claim 1 further comprising an in-line X-ray inspection machine for performing an X-ray inspection process on the PCB, said analysis engine being responsive to data and variables from the in-line X-ray inspection machine and providing feedback signals to the in-line X-ray inspection machine for self-correction purposes.

7. The system according to claim 1 further comprising an in-circuit testing machine for performing electrical testing on the PCB, said analysis engine being responsive to data and variables from the in-circuit testing machine and providing feedback signals to the in-circuit testing machine for self-correction purposes.

8. The system according to claim 1 wherein the process data and variables provided to the engine by the screen printer includes solder paste type, cleaning cycle stroke and screen printer parameters, said engine providing pressure adjustments, squeegee changes and stencil cleaning information to the screen printer that are determined from upstream processes and inspections for screen printing self-correction.

9. The system according to claim 1 wherein the process data and variables provided to the engine by the SPI sub-system includes solder paste offset measurements and pitch or resolution of components on the PCB, said engine providing information to the SPI sub-system that are determined from upstream processes and inspections for SPI self-correction.

10. The system according to claim 1 wherein the process data and variables provided to the engine by the pick-and-place machine includes ground, package and machine information, said engine providing change nozzle or feeder, adjust part definition, change placement position, optimize placement offset for better placement and perform maintenance information to the pick-and-place machine that are determined from upstream processes and inspections for pick-and-place self-correction.

11. The system according to claim 1 wherein the process data and variables provided to the engine by the first AOI sub-system includes component condition and component off-set measurements, said engine providing pre-flow program parameter adjustment settings to the first AOI sub-system that are determined from upstream processes and inspections for AOI self-correction.

12. A surface mount technology (SMT) manufacturing system for fabricating printed circuit board assemblies (PCBAs), said system comprising:
a screen printer for depositing solder paste on conductive solder pads on a printed circuit board (PCB);
a solder paste inspection (SPI) sub-system for inspecting the solder paste deposited on the PCB to identify defects;
a pick-and-place machine for placing circuit components on the solder paste;
a first automated optical inspection (AOI) sub-system for inspecting the PCB after the circuit components are placed on the PCB;
a reflow soldering oven for bonding component leads both electrically and mechanically to the pads on the PCB;
a second AOI sub-system for inspecting the PCB after the PCB has been to the reflow soldering oven;
an auto-insertion machine for inserting additional components on the PCB that are not able to be placed by the pick-and-place machine;
a wave solder machine for bulk soldering the PCB;
an in-line X-ray inspection machine for performing an X-ray inspection process on the PCB;
an in-circuit testing machine for performing electrical testing on the PCB; and
an artificial intelligence (AI)/machine learning (ML) analysis engine responsive to process data and variables from each of the screen printer, the SPI sub-system, the pick-and-place machine, the first AOI sub-system, the reflow soldering oven, the second AOI sub-system, the auto-insertion machine, the wave solder machine, the in-line X-ray inspection machine and the in-circuit testing machine and providing feedback signals to each of the screen printer, the SPI sub-system, the pick-and-place machine, the first AOI sub-system, the reflow soldering oven the second AOI sub-system, the auto-insertion machine, the wave solder machine, the in-line X-ray inspection machine and the in-circuit testing machine for self-correction purposes.

13. The system according to claim 12 wherein the analysis engine employs a self-learning Markov decision process (MDP) model that manages sequential decision process outcomes in which states and transitions are quantified in calculated rewards during the transition between two Markov states and provides multi-agent reinforcement learning.

14. The system according to claim 12 wherein the process data and variables provided to the engine by the screen printer includes solder paste type, cleaning cycle stroke and screen printer parameters, said engine providing pressure adjustments, squeegee changes and stencil cleaning information to the screen printer that are determined from upstream processes and inspections for screen printing self-correction.

15. The system according to claim 12 wherein the process data and variables provided to the engine by the SPI sub-system includes solder paste offset measurements and pitch or resolution of components on the PCB, said engine providing information to the SPI sub-system that are determined from upstream processes and inspections for SPI self-correction.

16. The system according to claim 12 wherein the process data and variables provided to the engine by the pick-and-place machine includes ground, package and machine information, said engine providing change nozzle or feeder, adjust part definition, change placement position, optimize placement offset for better placement and perform maintenance information to the pick-and-place machine that are determined from upstream processes and inspections for pick-and-place self-correction.

17. The system according to claim 12 wherein the process data and variables provided to the engine by the first AOI sub-system includes component condition and component off-set measurements, said engine providing pre-flow program parameter adjustment settings to the first AOI sub-system that are determined from upstream processes and inspections for AOI self-correction.

18. A surface mount technology (SMT) manufacturing system for fabricating printed circuit board assemblies (PCBAs), said system comprising:
a plurality of devices for fabricating a printed circuit board (PCB); and
an artificial intelligence (AI)/machine learning (ML) analysis engine responsive to process data and variables from each of the plurality of devices and providing feedback signals to each of the plurality of devices for self-correction purposes.

19. The system according to claim 18 wherein the analysis engine employs a self-learning Markov decision process (MDP) model that manages sequential decision process outcomes in which states and transitions are quantified in calculated rewards during the transition between two Markov states and provides multi-agent reinforcement learning.

20. The system according to claim 18 wherein the plurality of devices include a screen printer for depositing solder paste on conductive solder pads on the PCB, a solder paste inspection (SPI) sub-system for inspecting the solder paste deposited on the PCB to identify defects, a pick-and-place machine for placing circuit components on the solder paste, an automated optical inspection (AOI) sub-system for inspecting the PCB after the circuit components are placed on the PCB, and a reflow soldering oven for bonding component leads both electrically and mechanically to the pads on the PCB.
